(19) United States
(12) Patent Application Publication
(10) Pub. No.: US 2020/0306110 A1
(43) Pub. Date: Oct. 1, 2020

(54) A PACKAGING UNIT FOR ABSORBENT ARTICLES AND A METHOD OF MANUFACTURING A PACKAGING UNIT

(71) Applicant: Essity Hygiene and Health Aktiebolag, Goteborg (SE)

(72) Inventors: Anders Silfverstrand, GÖTEBORG (SE); Maria Swedberg, GÖTEBORG (SE)

(73) Assignee: Essity Hygiene and Health Aktiebolag, GÖTEBORG (SE)

(21) Appl. No.: 16/314,234

(22) PCT Filed: Jul. 5, 2016
(86) PCT No.: PCT/EP2016/065862

§ 371 Date: Dec. 28, 2018

(51) Int. Cl. A61F 13/551; B65D 75/20

(52) U.S. Cl. CPC A61F 13/5514; B65D 75/20

(57) ABSTRACT

A packaging unit for an absorbent article is formed from a sheet of material and includes a longitudinal center line, a transversal center line, a first longitudinal edge zone, a second longitudinal edge zone, a first transversal edge zone and a second transversal edge zone. At least one of the first longitudinal edge zone, the second longitudinal edge zone, the first transversal edge zone or the second transversal edge zone includes at least one zone provided with surface protrusions integrally formed from the material of the sheet of material. The sheet of material includes at least one landing zone able to engage with the surface protrusions to provide a fastening mechanism when said sheet of material is rolled or folded. A method of manufacturing a packaging unit and a method to close a packaging unit holding an absorbent article are also disclosed.

### CROSS REFERENCE TO RELATED APPLICATIONS

This is a U.S. National Phase application of PCT/EP2016/065862, filed Jul. 5, 2016, the disclosure of which is incorporated herein by reference in its entirety for all purposes.

### TECHNICAL FIELD

The invention relates to a packaging unit for absorbent articles. The present invention also relates to a method of manufacturing a packaging unit for absorbent articles from a sheet of material.

### BACKGROUND

Absorbent articles, such as diapers, incontinence products, sanitary napkins, panty liners, and tampons are commonly packaged individually, as for example, in an easy wrap or a single wrap. Individual packages facilitate hygienic carrying of single articles for future use for example in a handbag. The edges of the individual packages are often sealed by means of ultrasonic welding or heat welding. Further, the packaging units are often used both as a means for packaging an unused article and for disposal of the used article.

It is desirable that used articles of this kind can be disposed discretely and hygienically. This may be particularly important when the user lacks the possibility to dispose the used article immediately after the used article has been replaced, for example when there is no waste bin available in the toilet area. In this case, the user may need to put the used article for example in a handbag or backpack, which requires that the package is tightly sealed in order to avoid staining and odor.

To solve the problem of providing a single wrap that may be both used for packaging of a new and unused absorbent article and for safe and hygienic disposal of used absorbent articles, the use of a resealable adhesive has been shown to be useful. WO2013/162430 discloses such a packaging unit in the form of a wrap that can be used both for packaging of a new and unused absorbent article and for safe and hygienic disposing of used hygiene articles. The packaging unit comprises a sheet provided with an adhesive applied in a pattern, for example in a chessboard pattern, for closing and sealing the packaging unit. By applying the adhesive in an intermittent pattern contact between different adhesive areas is avoided. The use of adhesive has a number of drawbacks. If you close the packaging unit incorrectly the packaging unit can not be re-opened or adjusted. By trying to re-open or adjust, the sheet is damaged so that the packaging can not be sealed properly. If two adhesive areas of the intermittent pattern nevertheless come in contact with each other it is not possible to re-open the packaging unit without destroying the sheet.

The application of the intermittent adhesive pattern requires a complex and expensive method. The use of an adhesive increases the manufacturing cost, may lead to contamination of the manufacturing equipment and increases the maintenance of the equipment. Furthermore the adhesive may cause an unpleasant odor. As the adhesive is applied at an elevated temperature, a relative high weight of sheet material is required to avoid deformation of the sheet material. Packaging units known in the art may require two different adhesives: a first adhesive, preferably applied at the edges of the sheet, for closing and reopening the package and a second adhesive, preferably applied at the center of the sheet, to attach the absorbent article to the sheet. The second adhesive is transferred to the absorbent article, which allows the absorbent article to be attached to underwear by the second adhesive when the article is removed from the packaging. The center of the sheet of material is preferably provided with a silicone coating, which allows the second adhesive to move from the sheet of material. The application of the silicone coating at the center of the sheet further complicates the production process.

Thus, there is still a need for further improvements of packaging units so as to provide a packaging for packaging a new absorbent article and for keeping and disposing of the used absorbent article.

### SUMMARY

It is an object of the present disclosure to provide a packaging unit which can be used both for packaging a new absorbent article and for safe and hygienic disposing of the used absorbent article.

It is another object of the present disclosure to provide a packaging unit allowing to form a tight package both for a new absorbent article and for a used absorbent article.

It is a further object to provide a packaging unit allowing to keep an absorbent article clean prior to use as well allowing to eliminate the risk of staining and odor when a used article is packaged.

It is still a further object to provide a packaging unit that is easy to unfold and reseal and that is aesthetically appealing.

It is another object of the present invention to provide a method to form a packaging unit from a sheet of material.

According to a first aspect of the disclosure a packaging unit for an absorbent article is provided. The packaging unit according to the present disclosure is formed from a sheet of material. The sheet of material has an inner surface and an outer surface. The sheet has a longitudinal direction and a transversal direction perpendicular to the longitudinal direction and has a longitudinal center line, a transversal center line, a first longitudinal edge zone, a second longitudinal edge zone, a first transversal edge zone and a second transversal edge zone. At least one of the first longitudinal edge zone, the second longitudinal edge zone, the first transversal edge zone and the second transversal edge zone comprises at least one zone provided with surface protrusions. The surface protrusions are integrally formed from the material of the sheet. The sheet has furthermore at least one landing zone able to engage with the surface protrusions to provide a fastening mechanism when the sheet is rolled or folded. Preferably the at least one landing zone is able to releasably engage with the surface protrusions to provide a fastening mechanism when the sheet is rolled or folded.

The surface protrusions are preferably obtained by a softening and forming operation, for example by applying energy to at least one of the first longitudinal edge zone, the second longitudinal edge zone, the first transversal edge zone and the second transversal edge zone of the sheet of material to locally soften at least one of the first longitudinal edge zone, the second longitudinal edge zone, the first transversal edge zone or the second transversal edge zone. The surface protrusions are for example obtained by using a method as described in DE10102501 or as described in U.S. Pat. No. 8,784,722.

For the purpose of this disclosure “absorbent article” refers to consumer products capable of absorbing and containing fluids. A particular group of absorbent articles comprise consumer products capable of absorbing and containing body exudates, and more specifically, refers to products which are placed against or in proximity to the body of the wearer to absorb and contain the various exudates discharged from the body. Absorbent articles include, but are not limited to napkins, for example intended for wiping, sanitary napkins, panty liners, diapers, panty diapers, tampons and incontinence devices.

The term “inner surface” refers to the surface of the packaging unit facing the absorbent article inside the packaging unit. The term “outer surface” refers to the surface opposite to the inner surface, i.e. the surface facing the ambient.

The term “edge zone” refers to the portion of the sheet of material adjacent to the edges of the sheet of material. The width of an edge zone may vary and ranges for example between 2 and 40 mm.

The term “longitudinal” or “longitudinally” refers to the length or lengthwise direction of the sheet. Consequently, a “longitudinal edge zone” is extending along the longitudinal center line. The term “transversal” or “transversally” refers to the width or widthwise direction and corresponds with the direction perpendicular to the longitudinal direction. Consequently, a “transversal edge zone” is extending along the transversal center line.

In a first group of packaging units according to the present disclosure the first longitudinal edge zone or the second longitudinal edge zone comprise(s) at least one zone provided with surface protrusions. In preferred embodiments of the first group of packaging units both the first longitudinal edge zone and the second longitudinal edge zone comprise at least one zone provided with surface protrusions.

In a second group of packaging units the first transversal edge zone and/or the second transversal edge zone comprise(s) at least one zone provided with surface protrusions. In preferred embodiments of the second group of packaging units both the first transversal edge zone and the second transversal edge zone comprise at least one zone provided with surface protrusions.

In a third group of packaging units at least one of the first longitudinal edge zone or the second longitudinal edge zone and at least one of the first transversal edge zone or the second transversal edge zone comprise at least one zone provided with surface protrusions. The first longitudinal edge zone, the second longitudinal edge zone, the first transversal edge zone and/or the second transversal edge zone may comprise one zone provided with surface protrusions or may comprises a number of zones provided with surface protrusions, for example two, three, four or five zones provided with surface protrusion.

In case the first longituditial edge zone, the second longitudinal edge zone, the first transversal edge zone and/or the second transversal edge zone may comprise one zone provided with surface protrusions, such zone may extend over a part of the length of the first longitudinal edge zone, the second longitudinal edge zone, the first transversal edge zone and/or the second transversal edge zone or over the full length or substantially the full length of the first longitudinal edge zone, the second longitudinal edge zone, the first transversal edge zone and/or the second transversal edge zone.

The surface protrusions preferably protrude from the inner surface of the sheet of material. Possibly the surface protrusions protrude from the outer surface of the sheet of material or the surface protrusions protrude from both the inner surface and the outer surface of the sheet of material.

The surface protrusions can have any shape. Preferred examples of surface protrusions comprise pins, for example straight pins, angled pins, curved pins, tapered pins, limbed or multi-limbed pins, hooks, limbed or multi-limbed hooks, mushroom shaped protrusions and palm tree shaped protrusions. The surface protrusions may have any type of cross-section such as round, oval, square, rectangular, polygonal. The surface projections preferably have a solid core.

Within one zone of surface protrusions all surface protrusions may have the same shape. Alternatively one zone may comprise several different shapes of surface protrusions.

When a packaging unit has several zones provided with surface protrusion, the different zones can have the same shapes of surface protrusions or the different zones can have different shapes of surface protrusions.

The number of surface protrusions ranges preferably between 100 and 500 surface protrusions per cm², and more preferably between 166 and 408 surface protrusions per cm².

Within one zone of surface protrusions the number of surface protrusions per cm² can be constant or can vary over the zone.

When a packaging unit has several zones provided with surface protrusion, the different zones can have the same number of surface protrusions per cm² or the different zones can have a different number of surface protrusions per cm².

The sheet of material has at least one landing zone. As landing zone any type of zone that is able to engage and preferably able to releasably engage with the at least one zone provided with surface protrusions can be considered. A landing zone can be a zone attached to the sheet of material. Examples comprise a loop patch or a woven or non-woven patch attached to the sheet of material. The loop patch or woven or non-woven patch is for example attached to the sheet of material by gluing, melting or stitching. Alternatively the sheet of material as such can act as a landing zone. A preferred example of a sheet of material that as such can act as a landing zone comprises a non-woven structure or a woven structure or a sheet of material provided with protrusions, for example provided with hooks.

Preferably a zone provided with surface protrusions (either a longitudinal or transversal zone) and its cooperating landing zone has a mechanical interaction providing a sealing strength ranging between 0.5 N and 3.5 N. The sealing strength is determined by measuring the force required to open the seal obtained by a zone provided with surface protrusions and its cooperating landing zone. The force is measured by using a tensile tester.

In preferred embodiments, the sheet of material comprises one or more multilayered zones. The one or more multilayered zones is/are preferably provided with the above mentioned surface protrusions. By providing the sheet of material with one or more multilayered zones, more material is present in the multilayered zone to provide the surface protrusions. The surface protrusions are preferably obtained by a softening and forming operation, for example by applying energy to the at least one multilayered zone of the sheet of material to locally soften the at least one multilayered zone. By subjecting the multilayered zone to the energy, the different layers of the multilayered zone are connected and the multilayered zone is provided with surface protrusions in the same operation.

Preferably at least one of the first longitudinal edge zone, the second longitudinal edge zone, the first transversal edge zone or the second transversal edge zone comprise a multilayered zone.

A multilayered zone is for example obtained by folding the sheet of material towards the longitudinal center line to form a longitudinal multilayered edge zone or by folding the sheet of material towards the transversal center line to form a transversal multilayered edge zone.

In alternative embodiments the longitudinal multilayered zone is obtained by applying, for example gluing, melting or stitching, an additional layer, for example a strip. The additional layer is for example applied along one or both longitudinal edges and/or along one or both transversal edges.

The sheet of material may be provided with a first longitudinal multilayered edge zone at the first longitudinal edge zone and/or with a second longitudinal multilayered edge zone at the second longitudinal edge zone.

In alternative embodiments the sheet of material may be provided with a first transversal multilayered edge zone at the first transversal edge zone and/or with a second transversal multilayered edge zone at the second transversal edge zone.

A sheet of material can be provided with both longitudinal multilayered zone or zones and transversal multilayered zone or zones.

The sheet of material of the disclosed packaging unit may comprise a single layer or may comprise multiple layers for example a laminate comprising for example two or three layers. In case the sheet comprises multiple layers the different layers can be the same or can be different. The layer or layers may comprise a polymer film or foil, a coated film or foil, for example a polymer coated film or foil, a textile substrate such as a woven structure or a non-woven substrate or a coated textile substrate, for example a polymer coated textile structure.

The sheet of material comprises preferably a thermoplastic material. Examples of thermoplastic materials comprise polyamide, polyolefin such as polypropylene and polyethylene, polystyrene such as styrene-isoprene-styrene (SIS), styrene-butadiene-styrene (SBS), styrene-ethylene-butadiene-styrene (SEBS), acrylonitrile-butadiene-styrene (ABS), polyester, polycarbonate, polyvinyl chloride (PVC), polyetherester, polyetheramine and blends thereof.

The thermoplastic material may be modified or reinforced for example with fillers, fibers, flame retardants, colorants, etc.

The geometrical shape of the sheet can vary for example depending on the type of absorbent article to be packaged. The sheet may be circular or substantially circular, triangular or substantially triangular, square or substantially square, rectangular or substantially rectangular, or any other shape suitable for the absorbent article to be packaged. Preferred shapes are square or substantially square shapes and rectangular or substantially rectangular shapes.

The disclosed packaging unit has preferably a first packaging function as a packaging wrap for a non-used absorbent article, and a second disposal function as a disposal wrap for a used article.

An advantage of the disclosed packaging unit is that it provides the possibility of forming a tight package both for a new and a used article, thus keeping the new article sanitary and clean prior to use, and eliminating the risk of staining and odor when a used article is packaged. The packaging unit is easy to unfold and reseal. Furthermore, the packaging unit is preferably aesthetically appealing and provides an easy and intuitive identification of the disposal function of the packaging unit.

In a preferred embodiment the packaging unit for an absorbent article comprises at least one folding axis. The at least one folding axis is preferably parallel or substantially parallel with the transversal center line. The folding axis divides the sheet of material into a first region and a second region. Both the first region and the second region have a first longitudinal edge zone and a second longitudinal edge zone. Preferably at least one of the first or the second longitudinal edge zones of the first region comprises at least one zone provided with surface protrusions, whereas at least one of the first or the second longitudinal edge of the second region is provided with at least one landing zone. When the absorbent article is folded the at least one zone provided with surface protrusions of the first region is engaging (preferably releasably engaging) with the at least one landing zone of the second region.

It can be preferred that both the first and the second longitudinal edge zones of the first region comprise at least one zone provided with surface protrusions and that both the first and the second longitudinal edge zone of the second region comprise at least one landing zone.

Alternatively it can be preferred that one of the first longitudinal edge zone of the first region comprises at least one zone with surface protrusions while the second longitudinal edge zone of the first region comprises at least one landing zone, and that the first longitudinal edge zone of the second region comprises at least one landing zone while the second longitudinal edge zone comprises at least one zone provided with surface protrusions.

In another preferred embodiment the packaging unit for an absorbent article comprises more than one folding axis, for example two folding axes. The folding axes are preferably parallel or substantially parallel with the transversal center line. The folding axes divide the sheet of material into a first region, a second region and a third region.

According to a second aspect of the disclosure a method of manufacturing a packaging unit for absorbent articles as described above is provided. The method comprises the steps of
providing a sheet of material, the sheet of material having an inner surface and an outer surface, the sheet of material having a longitudinal center line, a transversal center line, a first longitudinal edge zone, a second longitudinal edge zone, a first transversal edge zone and a second transversal edge zone, the first longitudinal edge zone and the second longitudinal edge zone extending along the longitudinal center line, the first transversal edge zone and the second transversal edge zone extending along the transversal center line,
providing a forming die having a surface provided with a plurality of cavities, at least some of the cavities having a shape to produce surface protrusions,
bringing the inner surface or the outer surface of the sheet of material in contact with the first surface of the forming die;
applying energy to the sheet of material to locally soften at least one of the first longitudinal edge zone, the second longitudinal edge zone, the first transversal edge zone or the second transversal edge zone in such a way that material of the sheet of material of at least the first longitudinal edge zone, the second longitudinal edge zone, the first transversal edge zone or the second transversal edge zone is forced into the cavities to form surface protrusions at the inner surface and/or at the outer surface of the sheet of material.

Preferably the energy is applied by means of a source selected of a group consisting of induction heating, ultrasonic vibrations, micro waves, radio waves, infrared waves, a laser beam and an electron beam.

The forming die comprises preferably a roll provided with a plurality of cavities.

The surface protrusions are for example obtained by using a method as described in DE10102501 or as described in U.S. Pat. No. 8,784,722.

It can be preferred to apply a mask between the sheet of material and the forming die to provide the sheet of material with surface protrusions in a pattern.

In preferred embodiments energy is applied to locally soften both the first longitudinal edge zone and the second longitudinal edge zone in such a way that material of the sheet of material of both the first longitudinal edge zone and the second longitudinal edge zone is forced into the cavities to form surface protrusions at the inner surface and/or at the outer surface of the sheet of material.

In other preferred embodiments energy is applied to locally soften both the first transversal edge zone and the second transversal edge zone in such a way that material of the sheet of material of both the first transversal edge zone and the second transversal edge zone is forced into the cavities to. form surface protrusions at the inner surface and/or at the outer surface of the sheet of material.

It is clear that it is also possible to apply energy to locally soften the longitudinal edge zone(s) and the transversal edge zone(s) simultaneously in such a way that material of the sheet of material of the transversal edge zone(s) and the transversal edge zone(s) is forced into the cavities to form surface protrusions at the inner surface and/or at the outer surface of the sheet of material.

In case it is desired to provide both the inner surface and the outer surface of the packaging unit with surface protrusion it is preferred that the inner surface of the sheet of material is brought in contact with a first surface of a first forming die and that the outer surface of the sheet is brought in contact with a first surface of a second forming die. This can be realized in one step or alternatively in two subsequent steps.

According to a third aspect of the disclosure a method to close a packaging unit holding an absorbent article is provided. The method comprises the steps of
providing a sheet of material, the sheet of material having an inner surface and an outer surface, the sheet of material having a longitudinal center line, a transversal center line, a first longitudinal edge zone, a second longitudinal edge zone, a first transversal edge zone and a second transversal edge zone, the first longitudinal edge zone and the second longitudinal edge zone extending along the longitudinal center line, the first transversal edge zone and the second transversal edge zone extending along the transversal center line, at least one of the first longitudinal edge zone, the second longitudinal edge zone, the first transversal edge zone or the second transversal edge zone comprising at least one zone provided with surface protrusions extending from said inner surface and/or from said outer surface, said surface protrusions being integrally formed from said sheet of material, the sheet of material being provided with at least one landing zone;
applying an absorbent article either a used or an unused absorbent article on a surface, on the inner surface or on the outer surface, of said sheet of material,
folding or rolling said sheet of material comprising said absorbed article applied on said sheet of material such that said at least one zone provided with surface protrusions is brought in contact with said at least one landing zone whereby said surface protrusions of said at least one zone provided with surface protrusions engage with said at least one landing zone.

In a preferred embodiment the surface protrusions of the at least one landing zone releasably engage with the at least one landing zone.

### BRIEF DESCRIPTION OF THE DRAWINGS

Embodiments of the invention will now be described into more detail with reference to the accompanying drawings of which

FIG. 1A shows a first embodiment of a packing unit according to the present invention in its open state;

FIG. 1B shows the packaging unit of FIG. 1A in its folded state;

FIG. 1C shows the packaging unit of FIG. 1A in its rolled state;

FIG. 2A shows a second embodiment of a packing unit according to the present invention in its open state;

FIG. 2B shows the packaging unit of FIG. 2A in its folded state;

FIG. 2C shows the packaging unit of FIG. 2A in its rolled state;

FIG. 3A shows a third embodiment of a packing unit according to the present invention in its open state;

FIG. 3B shows the packaging unit of FIG. 3A in its folded state;

FIG. 4A shows a fourth embodiment of a packing unit according to the present invention having multilayered zones in its open state;

FIG. 4B shows the cross-section along a transversal line of a packing unit shown in FIG. 4A before subjected to a softening and forming operation;

FIG. 4C shows the folded packaging unit shown in FIG. 4B subjected to a softening and forming operation;

FIG. 5 shows a packing unit in its open state provided with an absorbent article;

FIG. 6 shows an embodiment how to provide a packaging unit as shown in FIGS. 1 to 5 with surface protrusions

### DETAILED DESCRIPTION OF PARTICULAR EMBODIMENTS

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn to scale for illustrative purposes. The dimensions and the relative dimensions do not correspond to actual reductions to practice of the invention.

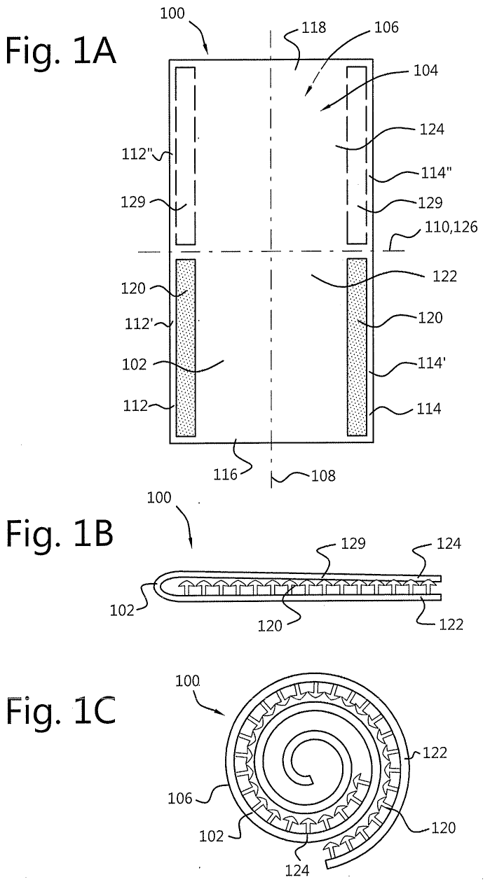

FIG. 1A shows a packing unit 100 for an absorbent article according to the present invention in its open, unfolded state. FIG. 1B shows the packaging unit 100 shown in FIG. 1A in its folded state, FIG. 1C shows the packing unit 100 shown in FIG. 1A in its rolled state. The packaging unit 100 is formed from a sheet of material 102 and comprises an inner surface 104 and an outer surface 106. The sheet of material 102 has a longitudinal center line 108, a transversal center line 110, a first longitudinal edge zone 112, a second longitudinal edge zone 114, a first transversal edge zone 116 and a second transversal edge zone 118. The first longitudinal edge zone 112 and the second longitudinal edge zone 114 are extending parallel to the longitudinal center line 108. The first transversal edge zone 116 and the second transversal edge zone 118 are extending parallel to the transversal center line 110.

The sheet of material 102 comprises for example a non-woven material.

The packaging unit 100 has one folding axis 126. The folding axis 126 in the embodiment shown in FIG. 1A corresponds with the transversal center line 110. The folding axis 126 divides the sheet of material into a first region 122 and a second region 124.

The first region 122 has a first longitudinal edge zone 112′ and a second longitudinal edge zone 114′; the second region 124 has a first longitudinal edge zone 112″ and a second longitudinal edge zone 114″.

At least one of the first longitudinal edge zone 112′ of the first region 122 or the second longitudinal edge zone 114′ of the first region 122 is provided with at least one zone provided with surface protrusions 120. Preferably, as shown in FIG. 1A, both the first longitudinal edge zone 112′ of the first region 122 and the second longitudinal edge zone 114′ of the first region 122 are provided with at least one zone provided with surface protrusions 120. The surface protrusions 120 may for example comprise hooks. The surface protrusions 120 are integrally made of the sheet of material 102. The sheet of material 102 is subjected to a softening and forming process such that said surface protrusions 120 are being integrally made of said sheet of material 102.

The second region 124 is preferably provided with one or more landing zones so that when the sheet of material is folded as for example shown in FIG. 1B, the surface protrusions 120 of the first region 122 engage (preferably releasably engage) with the at least one landing zone of the second region 124.

The second region 124 in FIG. 1A is provided with two landing zones 129 for the surface protrusions 120 in the first longitudinal edge zone 112′ of the first region 122 and the second longitudinal edge zone 114′ of the first region 122. The non-woven material of the sheet of material 102 acts as a landing zone. Alternatively, loop patches or any other kind of landing zone material can be attached to the sheet of material 102. This may be advantageous, for example, if the sheet of material 102 is a plastic film. The loop patch or landing zone material is preferably arranged on at least one of the first longitudinal edge zone 112″ of the second region 122 or the second longitudinal edge zone 114″.

For the packaging function of a non-used absorbent article of a packaging unit 100 according to the present invention the sheet of material 102 is preferably folded as shown in FIG. 1B to wrap the non-used absorbent article. When folded the surface protrusions 120 engage with the landing zone 129.

For the disposal function of a used absorbent article of a packaging unit 100 according to the present invention the sheet of material 102 can be folded as shown in FIG. 1B or can be rolled as shown in FIG. 1C to wrap the used absorbent article dependent on the choice of the user. When rolled together the surface protrusions 120 engage with a further landing zone on the outer surface 106, which is the non-woven material of the sheet of material 102 or may be a separately attached landing zone.

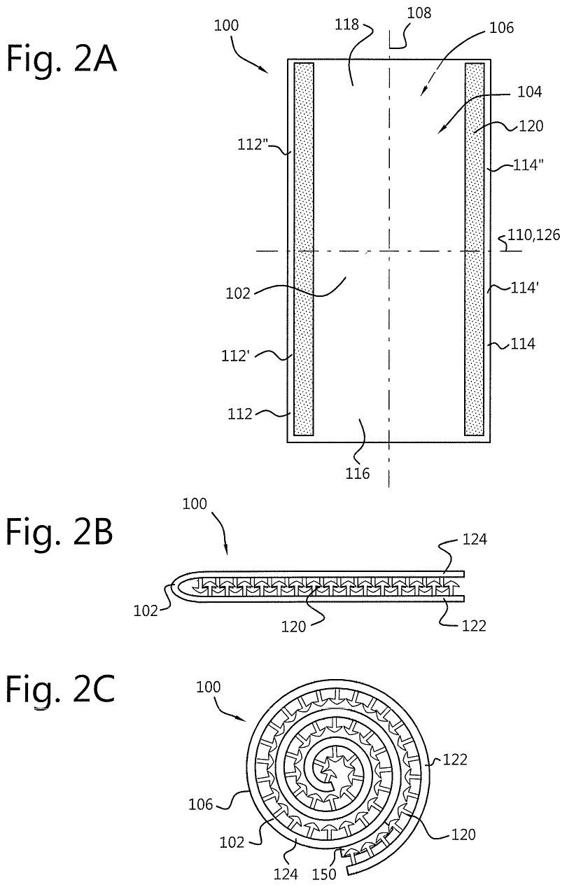

FIG. 2A shows a second embodiment of a packing unit 100 according to the present invention in its open, unfolded state. FIG. 2B shows the packaging unit 100 shown in FIG. 2A in its folded state, FIG. 2C shows the packing unit 100 shown in FIG. 2A in its rolled state. The packaging unit 100 of FIG. 2A is similar to the first embodiment shown in FIG. 1A but is different from the packaging unit 100 shown in FIG. 1A in that the sheet of material 102 comprises a polymer material such as a polymer film and in that the first longitudinal edge zone 112 and the second longitudinal edge zone 114 comprise zones provided with surface protrusions 120 that are extending over the major part of the length of the first longitudinal edge zone 112 and the second longitudinal edge zone 114. The zones provided with surface protrusions 120 engage with corresponding surface protrusions 120 of the polymer film, when the packaging unit 100 is folded as in FIG. 2B. In the rolled configurations some of the surface protrusions 120 engage a landing zone 150 arranged on the outer surface 106 of the sheet of material 102. Alternatively, all surface protrusions 120 or substantially all surface protrusions 120 may engage a landing zone on the outer surface 106 of the sheet of material 102.

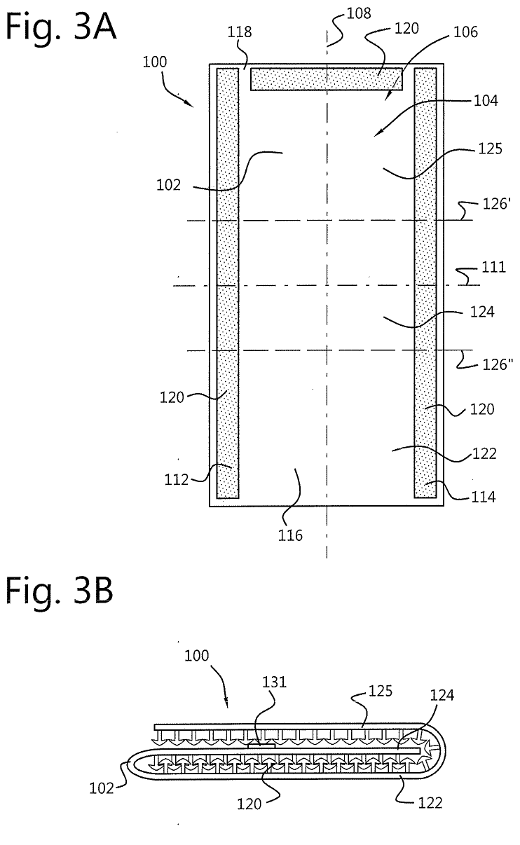

FIG. 3A shows a third embodiment of a packing unit 100 according to the present invention in its open, unfolded state. FIG. 3B shows the packaging unit 100 shown in FIG. 3A in its folded state. The packaging unit 100 has two folding axes 126′, 126″. The folding axes 126′, 126″ are parallel or substantially parallel with the transversal center line 110. The folding axis 126′, 126″ divide the sheet of material in a first region 122, a second region 124 and a third region 125. The first longitudinal edge zone 112 and the second longitudinal edge zone 114 comprise zones provided with surface protrusions 120 that are extending over the major part of the length of the first longitudinal edge zone 112 and the second longitudinal edge zone 114. One of the transversal edge zone, for example the second transversal edge zone 118 is provided with surface protrusions 120 that are extending over the major part of the length of the second transversal edge zone 118.

In addition the sheet of material can be provided with an additional means to fasten opposed surfaces for example by a fastening means 131 applied at the outer surface of the sheet of material 102. Such fastening means 131 comprises for example a loop patch or hook patch applied at the outer surface of the sheet of material 102. Alternatively, they are formed by the same softening and forming process as the surface protrusions 120.

Another example of the additional fastening means may comprise a sticky tape. The sticky tape may be arranged to the outer surface for example at one end of the sheet of material 102 and when the sheet of material 102 is rolled or folded together part of the sticky tape may be used to attach the end of the sheet of material 102 to the outer surface of the sheet of material which will be next to it, i.e. to help keeping the sheet of material in a rolled or folded state.

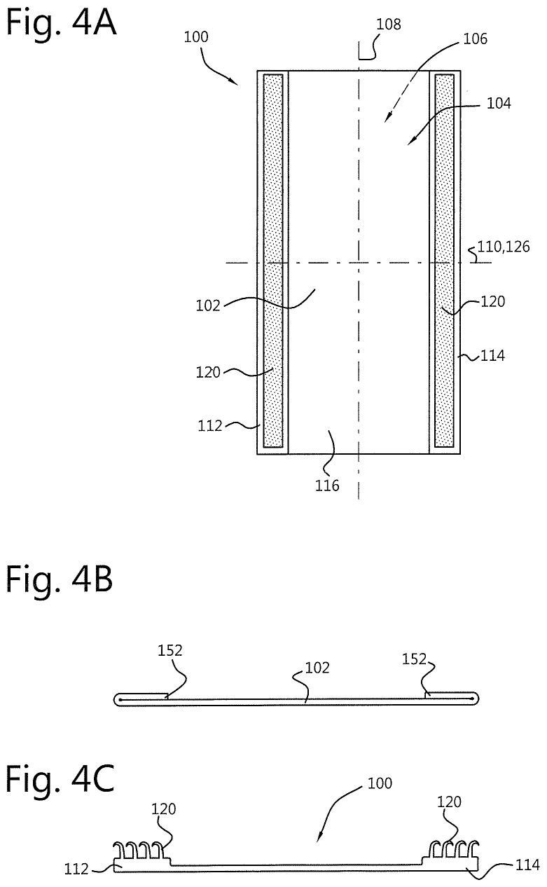

FIG. 4A shows a further embodiment of a packaging unit 100 according to the present invention in its open state. FIG. 4B shows the cross-section of the packaging unit 100 along a transversal line. The sheet of material 102 comprises at the first longitudinal edge zone 112 and at the second longitudinal edge zone 114 a multilayered zone obtained by folding a first portion 152 of the sheet of material 102 over the first longitudinal edge and the second longitudinal edge towards the longitudinal center line 108. FIG. 4c shows the folded portion of FIG. 4b once subjected to a softening and forming operation. By the softening and forming operation both layers are connected to each other and preferably form one layer of material. At least part of the double folded layer is provided with surface protrusions 120 by the same softening and forming operation. The dimensions and relative dimensions of the surface protrusions 120 are exaggerated and not drawn to scale for illustrative purposes. The height of the surface protrusions 120 typically range between 0.1 and 0.5 mm and is for example equal to 0.3 or 0.4 mm.

The folding can be any type of folding such as single folding, Z-shaped folding or double folding.

The multilayered zones are provided with surface protrusions 120, for example with hooks.

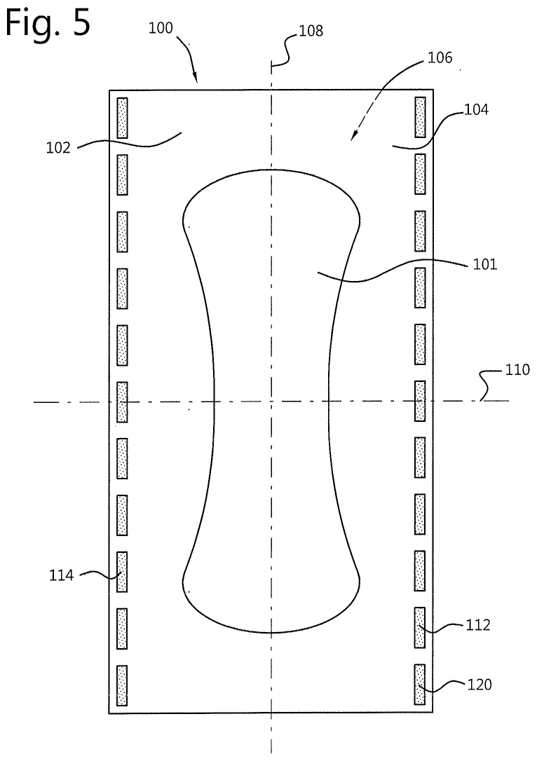

FIG. 5 shows a packaging unit 100 according to the present invention provided with an un-used absorbent article 101 as for example a sanitary napkin. The sheet of material 102 comprises at the first longitudinal edge zone 112 and at the second longitudinal edge zone 114 several zones provided with surface protrusions 120.

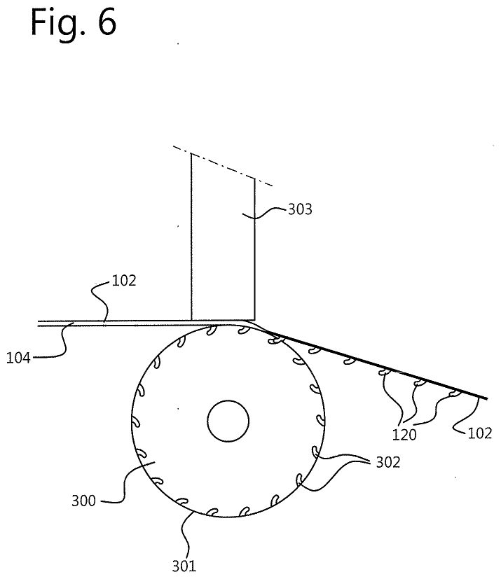

FIG. 6 shows one example how to provide the sheet of material 102 shown in FIGS. 1 to 5 with surface protrusions. FIG. 6 discloses a forming die 300, in form of a rotating moulding roll, which comprises a surface 301 provided a plurality of cavities 302, at least some of said cavities 302 comprise a shape to produce surface protrusions 120. The inner surface 104 of the sheet of material 102 is in contact with said surface 301 of said forming die 300. During operation a vibration source 303 is positioned in close proximity to the outer surface 301 of the rotating forming die 300. The vibration source 303 may be a vibrating ultrasonic horn. The vibration source 303 apply energy to the sheet of material 102. The material of the sheet of material 102 is softened and forced into said cavities 302 to provide said inner surface 104 of said sheet of material 102 with surface protrusions 120. If the sheet of material 102 is a folded portion, as for example shown in FIGS. 4a, 4b and 4c the energy from the vibration source 303 soften and form at least part of said sheet of material 102 in such a way that both layers connect and at the same time the material is forced into said cavities to provide said inner surface with surface protrusions 120.

While the present invention may be embodied in many different forms, a number of illustrative embodiments are described herein with the understanding that the present disclosure is to be considered as providing examples of the principles of the invention and such examples are not intended to limit the invention to preferred embodiments described herein and/or illustrated herein.

1. A packaging unit for an absorbent article, said packaging unit being formed from a sheet of material, said sheet of material, comprising an inner surface and an outer surface, said sheet of material having a longitudinal center line, a transversal center line, a first longitudinal edge zone, a second longitudinal edge zone, a first transversal edge zone and a second transversal edge zone, said first longitudinal edge zone and said second longitudinal edge zone extending along said longitudinal center line, said first transversal edge zone and said second transversal edge zone extending along said transversal center line, at least one of said first longitudinal edge zone, said second longitudinal edge zone, said first transversal edge zone or said second transversal edge zone comprise at least one zone provided with surface protrusions, said surface protrusions being integrally formed from said material of said sheet of material, said sheet of material comprising at least one landing zone able to engage with said surface protrusions to provide a fastening mechanism when said sheet of material is rolled or folded.

2. A packaging unit for an absorbent article according to claim 1, wherein said surface protrusions protrude from said inner surface and/or from said outer surface of said sheet of material.

3. A packaging unit for an absorbent article according to claim 1, wherein said first longitudinal edge zone and said second longitudinal edge zone have at least one zone provided with surface protrusions, said surface protrusions being integrally formed from said material of said sheet of material, said sheet of material comprising at least one landing zone able to engage with said surface protrusions to provide a fastening mechanism when said sheet is rolled or folded.

4. A packaging unit for an absorbent article according to claim 1, wherein said sheet of material is provided with at least one multilayered zone, said at least one multilayered zone being provided with said surface protrusions.

5. A packaging unit for an absorbent article according to claim 4, wherein said at least one multilayered zone is obtained by folding said sheet of material.

6. A packaging unit for an absorbent article according to claim 4, wherein said at least one multilayered zone is obtained by folding said sheet of material towards said longitudinal center line to form a longitudinal multilayered edge zone or by folding said sheet of material towards said transversal center line to form a transversal multilayered edge zone.

7. A packaging unit for an absorbent article according to claim 6, wherein said packaging unit comprises a first longitudinal multilayered edge zone and/or a second longitudinal multilayered edge zone and/or a first transversal multilayered edge zone and/or a second transversal multilayered edge zone.

8. A packaging unit for an absorbent article according to claim 4, wherein said at least one multilayered zone is obtained by applying an additional material along at least one of the first longitudinal edge zone, the second longitudinal edge zone, the first transversal edge zone or the second transversal edge zone.

9. A packaging unit for an absorbent article according to claim 1, wherein said sheet of material comprises a thermoplastic material, a woven structure or a non-woven structure.

10. A packaging unit for an absorbent article according to claim 1, wherein said surface protrusions comprise pins, straight pins, angled pins, curved pins, tapered pins, limbed or multi-limbed pins, hooks, limbed or multi-limbed hooks, mushroom shaped protrusions or -palm tree shaped protrusions.

11. A packaging unit for an absorbent article according to claim 1, wherein said at least one zone provided with surface protrusions and said at least one landing zone have a mechanical interaction providing a sealing strength ranging between 0.5 N and 3.5 N.

12. A packaging unit for an absorbent article according to claim 1, wherein said sheet has at least one folding axis, said folding axis dividing said sheet into a first region and a second region, both said first region and said second region having a first longitudinal edge zone and a second longitudinal edge zone, said first longitudinal edge zone and/or said second longitudinal edge zone of said first region being provided with at least one zone provided with said surface protrusions, said first longitudinal edge zone and/or said second longitudinal edge zone of said second region being provided with a landing zone able to engage with said surface protrusions of said first longitudinal edge zone and/or of said second longitudinal edge zone of said first region to provide a fastening mechanism when said sheet of material is rolled or folded.

13. A method of manufacturing a packaging unit for absorbent articles, said method comprising:
bringing an inner surface or an outer surface of a sheet of material in contact with a first surface of a forming die;
applying energy to said sheet of material to locally soften at least one of a first longitudinal edge zone, a second longitudinal edge zone, a first transversal edge zone or a second transversal edge zone, thereby forcing material of said sheet of material of at least one of said first longitudinal edge zone, said second longitudinal edge zone, said first transversal edge zone or said second transversal edge zone into cavities defined by the first surface of the forming die to form surface protrusions at said inner surface and/or at said outer surface of said sheet of material.

14. A method according to claim 13, wherein said energy is applied by a source selected from the group consisting of induction heating, ultrasonic vibrations, micro waves, radio waves, infrared waves, laser beams and electron beams.

15. A method to close a packaging unit holding an absorbent article, said method comprising:
providing a sheet of material, said sheet of material having an inner surface and an outer surface, the sheet of material having a longitudinal center line, a transversal center line, a first longitudinal edge zone, a second longitudinal edge zone, a first transversal edge zone and a second transversal edge zone, said first longitudinal edge zone and said second longitudinal edge zone extending along said longitudinal center line, said first transversal edge zone and said second transversal edge zone extending along said transversal center line, at least one of said first longitudinal edge zone, said second longitudinal edge zone, said first transversal edge zone or said second transversal edge zone comprising at least one zone provided with surface protrusions extending from said inner surface and/or from said outer surface, said surface protrusions being integrally formed from said sheet of material, said sheet of material being provided with at least one landing zone;
applying an absorbent article on said inner surface or said outer surface of said sheet of material,
folding or rolling said sheet of material comprising said absorbent article applied on said sheet of material, thereby bringing said at least one zone provided with surface protrusions in contact with said at least one landing zone whereby said surface protrusions of said at least one zone provided with surface protrusions engage with said at least one landing zone.
